(19) United States
(12) Patent Application Publication
(10) Pub. No.: US 2021/0161599 A1
(43) Pub. Date: Jun. 3, 2021

(54) STEERABLE TIP NEEDLE

(71) Applicant: CEDARS-SINAI MEDICAL CENTER, Los Angeles, CA (US)

(72) Inventors: Laith H. JAMIL, Bloomfield Hills, MI (US); Ali REZAIE, West Hollywood, CA (US)

(73) Assignee: Laith H. JAMIL, Bloomfield Hills, MI (US)

(21) Appl. No.: 17/047,250

(22) PCT Filed: Apr. 15, 2019
(86) PCT No.: PCT/US2019/027511

§ 371 Date: Oct. 13, 2020

Related U.S. Application Data
(60) Provisional application No. 62/657,665, filed on Apr. 13, 2018.

(51) Int. Cl. A61B 34/20; A61B 17/34; A61B 10/00; A61B 8/00; A61B 8/12

(52) U.S. Cl. CPC A61B 34/20; A61B 17/3478; A61B 10/0045; A61B 17/3403; A61B 2034/2063; A61B 8/12; A61B 8/565; A61B 2017/3413; A61B 8/445

(57) ABSTRACT

A device, a system, and a method for carrying out an endoscopic procedure in a patient's body using an endoscopic device comprising (a) a tube with a proximal end and a distal end, the distal end coupled to a handle and a proximal end configured to enter into the patient's body, (b) an ultrasound probe on the proximal end of the tube, positioned to emit sound waves to create a visual image of a target site inside the patient's body, and (c) a needle that is configured to be housed inside the tube, wherein the tube includes an internal cavity for a development of the needle.

### FIELD OF THE INVENTION

The present disclosure generally relates to a device and method for an endoscopic needle. More specifically, the disclosed devices and methods relate to novel endoscopic needles that include a steerable tip.

### BACKGROUND OF THE DISCLOSURE

Current endoscopic ultrasound needles have a rigid end and are used to sample lesions or access extraluminal areas of the patient's body such as the bile ducts, pancreatic ducts, gall bladder, small bowel, and the like. The purpose of these ultrasound needles is to either deliver injectable materials or to then access that organ with catheters, stents, or other devices. This would usually involve passing a wire through the needle to allow further access. Given the rigid nature of the needle tip, the wire can only be directed by rotating the wire or bouncing it off an opposing wall.

### SUMMARY OF THE DISCLOSURE

As such, it is challenging to direct endoscopic ultrasound needles in a certain direction once an organ such as a bile duct or pancreatic duct has been reached by the needle. The disclosed invention would address these problems by using a steerable tip or a tip that can be angled after it accesses an intended organ of a patient—making it easier to direct the wire or intended therapy in a certain direction within the accessed organ.

The following embodiments and aspects thereof are described and illustrated in conjunction with systems, compositions and methods that are meant to be exemplary and illustrative, not limiting in scope.

In an aspect of the present disclosure, an endoscopic device for use inside a patient's body is disclosed. The endoscopic disclosure includes a tube with a proximal end and a distal end, the distal end coupled to a handle and the proximal end configured to enter into the patient's body through the channel of an endoscopic ultrasound echoendoscope which has an ultrasound probe on the proximal end of the tube (scope), positioned to emit sound waves to create a visual image of a target site inside the patient's body; and a hole near or at the proximal end for a deployment of a needle (or a wire), wherein the tube includes an internal cavity.

The needle may include at least one of: flexible metal, carbon fiber, plastic, or any other material that is non-toxic inside a human body.

The needle, using the visual image for guidance, is directed to the target site. Once at the target site, the needle may be configured to carry out aspiration or suction at the target site in vivo.

The needle may include a hollow elbow positioned at or near the proximal end of the needle and is further configured to allow the needle to move freely with an assistance from the elbow. The elbow may include a swivel ball joint that is capable of rotating up to 270 degrees (or more) in any direction. During rotation of the elbow, the needle channel remains patent (or open). Multiple elbow joints with variable rotation angles and directions can be positioned proximal to the needle tip. A stiffening cannula can be placed inside the needle to avoid inadvertent rotation of the needle upon insertion into tissue. Depending on the indication of needle insertion, elbow joint can be placed more distal or proximal to the needle tip to have variable range of rotation.

The needle may include a proximal end that is configured to be deployed through the internal cavity of the tube to exit at the hole; and a distal end that is configured to be connected to a vacuum for suctioning at the target site. The needle tip at its proximal end may be steerable once inside the target site.

The needle may be steered by at least one of the following means: a force, an inner wire inside an internal tube on the needle that is configured to steer the needle based on physician's operation or automatically using a computer guided process, an electro-mechanical mechanism inside the needle that receives an instruction from a computer or a physician to change the needle's direction or angle, a hinge or any other mechanism inside the needle, or a combination thereof.

The needle may include a portion that is flexible at a mid-portion of the needle and a portion that is not flexible at the proximal end so that the mid-portion may change its shape upon application of the above described means. In some embodiments, the proximal end of the needle may be flexible as to allow the needle to change its directions.

The needle may include an internal cavity for a deployment of a needle at a patient's target site. The needle may also include a rod that includes a plastic or metal and is further configured to provide a stabilizing force to the needle so that the needle maintains its shape until the needle reaches a target site at which point the removable rod is removed from the needle so that the needle can be steered or flexibly moved. The handle may be configured to be connected to a machine for operations or may be manually operated by a physician. The handle may be configured to receive an instruction from a user (or a computer) and transmit said instruction to the needle and/or the guidewire such as, e.g., begin aspiration, deploy the guidewire/needle, carry out a medical procedure, move forward, backward, or sideways, and the like.

In an embodiment of the present disclosure, the endoscopic device as disclosed herein may be used for other treatments, such as, for example, vascular, radiology, and the like.

Another aspect of the present disclosure provides a method for using the endoscopic device including: inserting the endoscopic device into a body cavity during an endoscopic surgical procedure; inserting the needle (or wire) at a target site, e.g., an organ; steering the wire as needed; and carrying out aspiration via the wire in order to collect a sample from a patient's body. The method may further include re-puncturing the organ as needed. The needle may be configured to be steered to change angles and directions.

In yet another aspect of the present disclosure, a computer readable storage medium tangibly embodying a computer readable program code is provided, wherein the computer readable program code includes computer readable instructions which, when implemented, cause a computer to carry out the steps of a method as described herein.

### Preferred Embodiments

Embodiment 1. An endoscopic device for use in a patient's body, including a tube having an internal cavity with a proximal end and a distal end, the distal end coupled to a handle and a proximal end configured to enter into the patient's body; an ultrasound probe on the proximal end of the tube, positioned to emit sound waves to create a visual image of a target site inside the patient's body; and a needle that is configured to be housed inside the internal cavity of the tube and configured to be deployed from the internal cavity, wherein the needle is configured to steerable once the needle is placed at the target site.

Embodiment 2. The endoscopic device of embodiment 1, wherein the needle includes at least one of: flexible metal, carbon fiber, plastic, or any other material that is non-toxic inside a human body.

Embodiment 3. The endoscopic device of embodiment 1, wherein the needle includes a hollow elbow whereby the proximal end of the needle is configured to move freely along with a movement by the hollow elbow.

Embodiment 4. The endoscopic device of embodiment 3, wherein the hollow elbow is configured to be located at or near the proximal end of the needle.

Embodiment 5. The endoscopic device of embodiment 4, wherein the hollow elbow includes (a) a swivel ball joint that is capable of rotating up to 270 degrees in any direction, wherein during rotation of the elbow, needle channel remains patent, (b) multiple elbow joints with variable rotation angles and directions that are positioned proximal to the needle tip, and (c) a stiffening cannula configured to be placed inside the needle to avoid inadvertent rotation of the needle upon insertion into tissue, wherein depending on indication of needle insertion, the multiple elbow joints are placed more distal or proximal to the proximal end of the needle to have variable range of rotation.

Embodiment 6. The endoscopic device of embodiment 5, wherein the hollow elbow includes mechanistic features, such as, hinges, screws, fasteners, pulling mechanism, and other fastening mechanisms, that would allow the needle or a portion of the needle that protrudes from the hollow elbow to change its angle and/or direction as needed or controlled by a physician or a system computer.

Embodiment 7. The endoscopic device of embodiment 5, wherein the hollow elbow is further configured to be fixedly attached to the needle.

Embodiment 8. The endoscopic device of embodiment 1, wherein the ultrasound probe includes an ultrasound transducer that emits and detects the sound wave to create a visual image of a target site inside the patient's body.

Embodiment 9. The endoscopic device of embodiment 8, wherein the needle, using the visual image for guidance, is directed to the target site.

Embodiment 10. The endoscopic device of embodiment 9, wherein the needle includes: a proximal end that is configured to be deployed through the internal cavity of the tube; and a distal end that is configured to be connected to a vacuum for applying suction at the target site through the proximal end of the needle.

Embodiment 11. The endoscopic device of embodiment 9, wherein the needle is configured to carry out aspiration at the target.

Embodiment 12. The endoscopic device of embodiment 9, wherein the needle is steered by at least one of the following means: (a) an inner wire inside an internal tube of the needle that is configured to steer the needle based on physician's operation or automatically using a computer guided process, (b) a mechanical feature inside the needle that receive an instruction from a computer or a physician to change the needle's direction or angle, (c) a hinge or any other mechanism inside the needle, (d) a pulley system that directs the needle's movement, or (e) a combination thereof.

Embodiment 13. The endoscopic device of embodiment 12, wherein the needle includes a portion that is flexible at a mid-portion of the needle and a portion that is not flexible at the proximal end of the needle so that the mid-portion of the needle may change its shape upon steering the needle thereby changing the shape of the needle.

Embodiment 14. The endoscope device of embodiment 12, wherein the proximal end of the needle is configured to be flexible as to allow the needle to change its direction or angle.

Embodiment 15. The endoscopic device of embodiment 1, wherein the handle is configured to be connected to a machine for operations or manually operated by a physician.

Embodiment 16. The endoscopic device of embodiment 1, wherein the needle includes an internal cavity that is configured to fit a removable rod inside the needle's internal cavity so that the needle maintains its shape.

Embodiment 17. The endoscopic device of embodiment 16, wherein the removable rod is further configured to provide a stabilizing force to the needle so that the needle maintains its shape until the needle reaches a target site inside the patient at which point the removable rod is removed from the needle so that the needle can be steered or flexibly moved.

Embodiment 18. A method for using an endoscopic device inside a patient's body including: inserting the endoscopic device of embodiment 1 into a body cavity during an endoscopic surgical procedure; steering the needle; and carrying out an aspiration via the needle in order to collect a sample from the patient's body.

Embodiment 19. A system for carrying out an aspiration inside a patient's body including: an endoscopic device; a network; a monitor computer; a hosted server; and a database, wherein the network, the monitor computer, the hosted server, the database are all coupled to each other via communication links, and wherein the endoscopic device includes (a) a tube with a proximal end and a distal end, the distal end coupled to a handle and a proximal end configured to enter into the patient's body, (b) an ultrasound probe on the proximal end of the tube, positioned to emit sound waves to create a visual image of a target site inside the patient's body, and (c) a needle that is configured to be housed inside the tube, wherein the tube includes an internal cavity for a deployment of the needle.

Embodiment 20. The system of embodiment 19, wherein the endoscopic device, the monitor computer, the hosted server, and the database may each include a computer-readable medium tangibly embodying a computer readable program code having computer readable instructions which, when implemented, cause a computer to carry out the step of: inserting the endoscopic device into a body cavity during an endoscopic surgical procedure;

steering the needle; and carrying out an aspiration via the needle or the guidewire in order to collect a sample from the patient's body.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 illustrates an example of a system for an endoscopic aspiration that is constructed in accordance with the principles of this disclosure.

FIG. 2 illustrates an example of a method for using an endoscopic device that is constructed in accordance with the principles of this disclosure.

FIG. 3 illustrates an example of an endoscopic device with a hollow elbow that is constructed in accordance with the principles of this disclosure.

FIG. 4 illustrates another example of an endoscopic device with a hollow elbow that is constructed in accordance with the principles of this disclosure.

FIG. 5 illustrates another example of an endoscopic device with a hollow elbow that is constructed in accordance with the principles of this disclosure.

FIG. 6 illustrates yet another example of an endoscopic device with a hollow elbow that is constructed in accordance with the principles of this disclosure.

### DETAILED DESCRIPTION OF THE DISCLOSURE

The disclosure and the various features and advantageous details thereof are explained more fully with reference to the non-limiting implementations and examples that are described and/or illustrated in the accompanying drawings, and detailed in the following description. It should be noted that the features illustrated in the drawings are not necessarily drawn to scale, and features of one implementation may be employed with other implementations as any person skilled in the art would recognize, even if not explicitly stated herein. Descriptions of well-known components and processing techniques may be omitted so as to not unnecessarily obscure the implementations of the disclosure. The examples used herein are intended merely to facilitate an understanding of ways in which the disclosure may be practiced and to further enable those of skill in the art to practice the implementations of the disclosure. Accordingly, the examples and implementations herein should not be construed as limiting the scope of the disclosure.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application. The terms “patient” and “subject” are used interchangeably herein. These terms are intended to include all animal subjects, including mammals. Human patients/subjects are intended to be within the scope of the patients/subjects treated using the various embodiments of the inventive systems, apparatuses and methods described herein.

A term “wireless transmitter,” as used in this disclosure, means at least one of microwave, Infrared or RF module or a cellular/wireless modem and is configured to transmit data.

The term “coupled” means at least either a direct electrical connection between the connected items or an indirect connection through one or more passive or active intermediary devices. The term “circuit” means at least either a single component or a multiplicity of components, either active and/or passive, that are coupled together to provide a desired function. The term “signal” as used herein may include any meanings as may be understood by those of ordinary skill in the art, including at least an electric or magnetic representation of current, voltage, charge, temperature, data or a state of one or more memory locations as expressed on one or more transmission mediums, and generally capable of being transmitted, received, stored, compared, combined or otherwise manipulated in any equivalent manner.

Terms such as “providing,” “processing,” “supplying,” “determining,” “calculating” or the like may refer at least to an action of a computer system, computer program, signal processor, logic or alternative analog or digital electronic device that may be transformative of signals represented as physical quantities, whether automatically or manually initiated.

A “computer,” as used in this disclosure, means any machine, device, circuit, component, or module, or any system of machines, devices, circuits, components, modules, or the like, which are capable of manipulating data according to one or more instructions, to such as, for example, without limitation, a processor, a microprocessor, a central processing unit, a general purpose computer, a cloud, a super computer, a personal computer, a laptop computer, a palmtop computer, a mobile device, a tablet computer, a notebook computer, a desktop computer, a workstation computer, a server, or the like, or an array of processors, microprocessors, central processing units, general purpose computers, super computers, personal computers, laptop computers, palmtop computers, mobile devices, tablet computers, notebook computers, desktop computers, workstation computers, servers, or the like.

A “server,” as used in this disclosure, means any combination of software and/or hardware, including at least one application and/or at least one computer to perform services for connected clients as part of a client-server architecture. The at least one server application may include, but is not limited to, for example, an application program that can accept connections to service requests from clients by sending back responses to the clients. The server may be configured to run the at least one application, often under heavy workloads, unattended, for extended periods of time with minimal human direction. The server may include a plurality of computers configured, with the at least one application being divided among the computers depending upon the workload. For example, under light loading, the at least one application can run on a single computer. However, under heavy loading, multiple computers may be required to run the at least one application. The server, or any if its computers, may also be used as a workstation.

A “database,” as used in this disclosure, means any combination of software and/or hardware, including at least one application and/or at least one computer. The database may include a structured collection of records or data organized according to a database model, such as, for example, but not limited to at least one of a relational model, a hierarchical model, a network model or the like. The database may include a database management system application (DBMS) as is known in the art. The at least one application may include, but is not limited to, for example, an application program that can accept connections to service requests from clients by sending back responses to the clients. The database may be configured to run the at least one application, often under heavy workloads, unattended, for extended periods of time with minimal human direction. A “communications network,” as used in this disclosure, means a wired and/or wireless medium that conveys data or information between at least two points. The wired or wireless medium may include, for example, a metallic conductor link, a radio frequency (RF) communication link, an Infrared (IR) communication link, telecommunications networks, an optical communication link, internet (wireless and wired) or the like, without limitation. The RF communication link may include, for example, WiFi, WiMAX, IEEE 802.11, DECT, 0G, 1G, 2G, 3G, 4G, 5G or future cellular standards, Bluetooth, Bluetooth Low Energy, NFC, ultrasound, induction, laser (or similar optical transmission) and the like.

A “computer-readable storage medium,” as used in this disclosure, means any medium that participates in providing data (for example, instructions) which may be read by a computer. Such a medium may take many forms, including non-volatile media, volatile media, and transmission media. Non-volatile media may include, for example, optical or magnetic disks, flash memory, and other persistent memory. Volatile media may include dynamic random access memory (DRAM). Transmission media may include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium may include a “Cloud,” which includes a distribution of files across multiple (e.g., thousands of) memory caches on multiple (e.g., thousands of) computers.

Various forms of computer readable media may be involved in carrying sequences of instructions to a computer. For example, sequences of instruction (i) may be delivered from a RAM to a processor, (ii) may be carried over a wireless transmission medium, and/or (iii) may be formatted according to numerous formats, standards or protocols, including, for example, WiFi, WiMAX, IEEE 802.11, DECT, 0G, 1G, 2G, 3G or 4G cellular standards, Bluetooth, or the like.

A “network,” as used in this disclosure means, but is not limited to, for example, at least one of a local area network (LAN), a wide area network (WAN), a metropolitan area network (MAN), a personal area network (PAN), a campus area network, a corporate area network, a global area network (GAN), a broadband area network (BAN), a cellular network, the Internet, the cloud network, or the like, or any combination of the foregoing, any of which may be configured to communicate data via a wireless and/or a wired communication medium. These networks may run a variety of protocols not limited to TCP/IP, IRC, SSL, TLS, UDP, or HTTP.

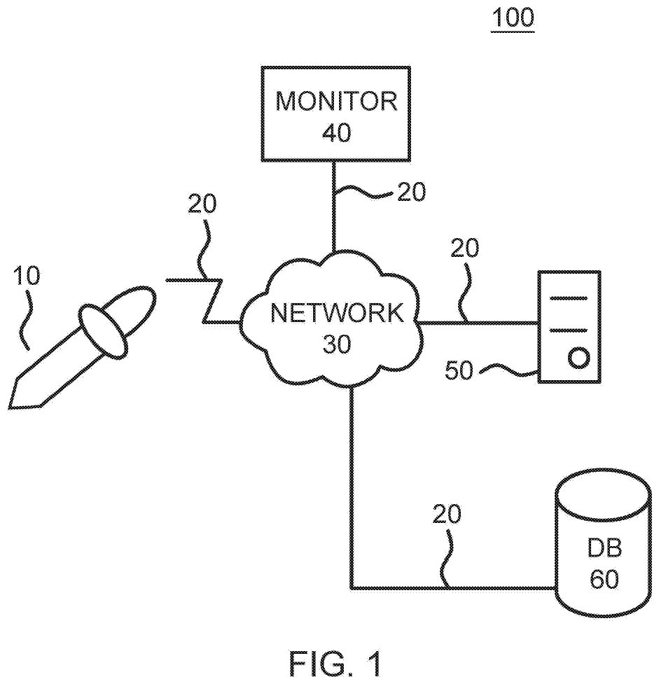

FIG. 1 shows an example of a system that is constructed according to the principles of the disclosure that provide wireless navigation of an endoscopic device to a treatment (or suction) site of a patient, and receiving and carrying out wireless transmission of instructions, such as, for example, begin aspiration, deploy balloon, take a biopsy, apply treatment, and the like. The system 100 includes at least one endoscopic device (or aspiration catheter) 10, a network 30, a monitor (e.g., a system manager) computer (or computing device) 40, a hosted server (or computer) 50, and a database 60, all of which may be coupled to each other via communication links 20. For instance, the hosted server 50 and database 60 may be connected to each other and/or the network 30 via one or more communication links 20. The endotracheal device 10 and the monitor computer 40 may be coupled to the network 30 via communication links 20. The endoscopic device 10 may be used by, for example, an authorized user (e.g., doctor, nurse, or the like) of a patient to whom the endoscopic device 10 is being used. Once the endoscopic device 10 collects/aspirates liquid in vivo, said liquid may then be transferred immediately to aerobic and/or anaerobic sterile tubes for microbiologic analysis.

The endoscopic device 10, the monitor computer 40, the hosted server 50, and the database 60 may each include a computer-readable medium including a computer program that may be executed to carry out the processes disclosed herein. The computer-readable medium may include a code section or code segment for performing each step disclosed herein.

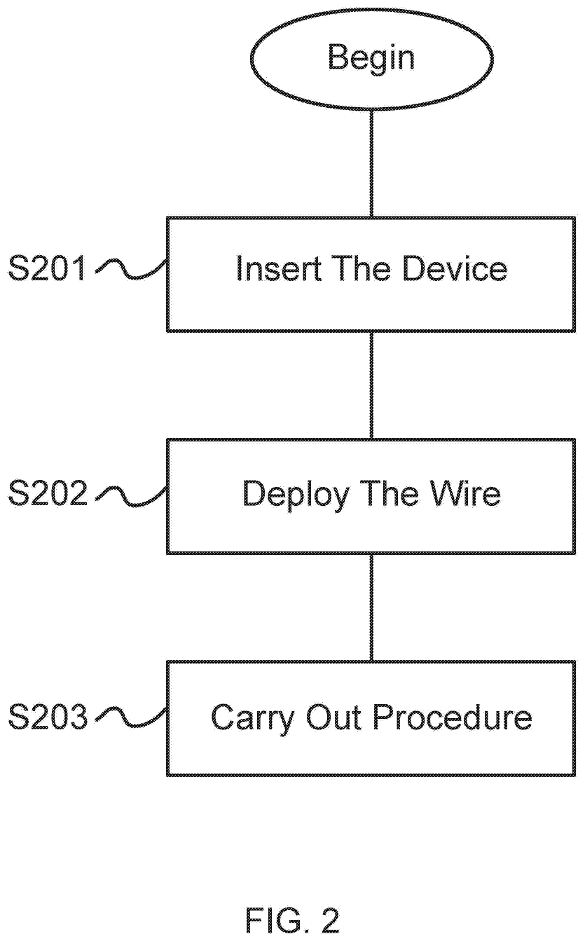

FIG. 2 illustrates an example of a method 200 for using an endoscopic device in accordance with the present disclosure. The method 200 includes inserting the endoscopic device into a body cavity (e.g., mouth) during an endoscopic surgical procedure (e.g., biopsy, aspiration, and the like) (Step 201); deploying a needle (or a wire) through the endoscopic device at a target site, e.g., an organ (Step 202); and carrying out a surgical procedure via the needle (Step 203).

In Step 201, the endoscopic device of the present invention may also be deployed for use in, e.g., gastrointestinal tract, small and large intestine, magnification endoscopy, bile duct, rectum, the respiratory tract, the ear, the urinary tract, the female reproductive system, and through a small incision. It may be used for medical procedures, such as, for example, pregnancy, plastic surgery, panendoscopy, orthopedic surgery, endodontic surgery, endoscopic endonasal surgery, and endoscopic spinal surgery, biopsy, transplantation, repair, and other surgical procedures.

In Step 201, the endoscopic device may include an ultrasound probe that is configured to guide the endoscopic device to a desired (or predetermined) site inside the patient through emitting sound waves to create the visual image of the target site in vivo. Alternatively (or additionally), the endoscopic device is configured to go through a channel of an endoscopic ultrasound machine/scope and is guided by the scope.

In Step 203, the surgical procedure(s) may include, for example, plastic surgery, panendoscopy, orthopedic surgery, endodontic surgery, endoscopic endonasal surgery, and endoscopic spinal surgery, biopsy, transplantation, repair, and other surgical procedures.

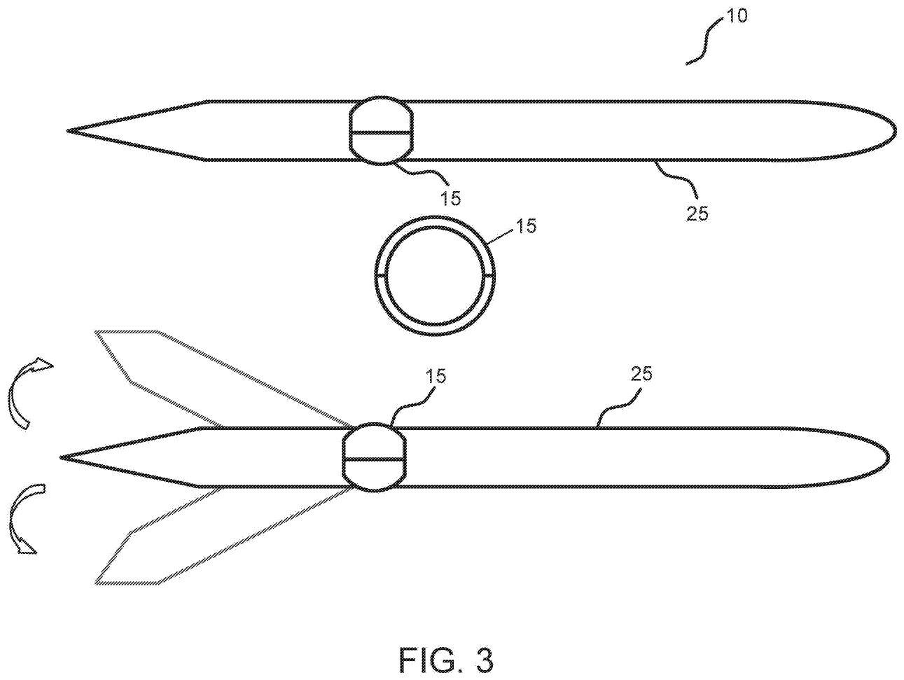

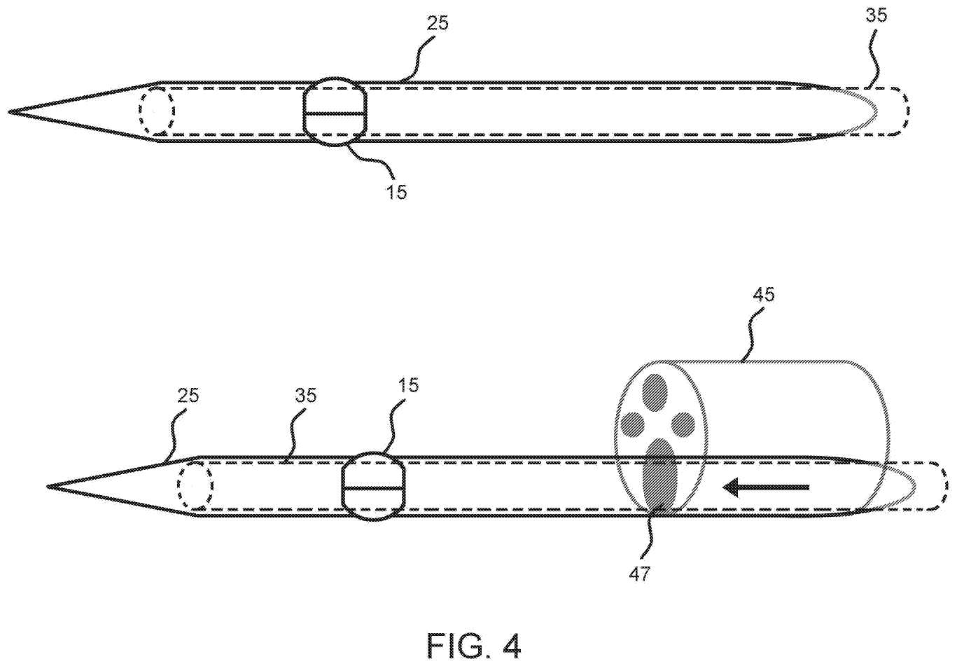

FIG. 3 illustrates an example of an endoscopic device 10 for use inside a patient's body in accordance with the principles of the present disclosure. Referring to FIGS. 3-4 concurrently, the endoscopic device 10 may be configured to be inserted through a tube (shown as tube 45 in, e.g., FIG. 4) with a proximal end and a distal end, the distal end coupled to a handle outside the patient's body and the proximal end configured to enter into the patient's body; and an ultrasound probe on the proximal end of the tube 45, positioned to emit sound waves to create a visual image of a target site inside the patient's body, wherein the tube 45 includes an internal cavity and a hole 47 for a deployment of a needle 25.

The needle 25 may be made of materials, such as, for example, flexible metal, carbon fiber, plastic, or any other material that is non-toxic inside a human body. The needle 25 may include other biodegradable or biocompatible material so that if the needle 25 is lost inside the body, it does not cause harm to the body. The needle 25 may include a proximal end that is configured to go through the internal cavity of the tube 45 to be deployed at the hole 47 once the tube 45 is at a predetermined or preferred site in vivo. The needle 25 may include a hollow elbow 15 that is positioned at or near the proximal end of the needle 25 and is further configured to allow the needle 25 to move freely with assistance from the elbow 15. The elbow 15 may include mechanistic features, such as, hinges, screws, fasteners, adhesives, and/or other fastening mechanisms, that would allow the needle 25 or a portion of the needle 25 (e.g., tip) that protrudes from the elbow 15 to change its angle and/or direction as needed or controlled by a physician. Alternatively, such movements and other methods as disclosed herein may be controlled by a computer.

The ultrasound probe may be attached to the tube 45 which may be an endoscopic ultrasound machine/scope (such as, e.g., prosound F75 Hitachi ultrasound system or curved linear array ultrasound processors) from which the needle 25 may advance through the internal channel 47 of the tube 45. The ultrasound probe may include an ultrasound transducer that emits and detects the sound waves to create the visual image of the target site in vivo. The needle 25, using the visual image for guidance and traveling through the internal cavity of the tube 45, is directed to the target site. Once at the target site, the needle 25 may be configured to extend out of the tube 45 through the internal channel (hole) 47 to carry out, e.g., aspiration or suction, at the target site in vivo. The needle 25 may extend out of the tube 45 via, e.g., using a pushing mechanism at the distal end of the tube 45. The needle 25 may also be configured to be compressed inside the tube 45 and extended once at the target site via mechanical means, such as, e.g., Additionally (or alternatively), the needle 25 may carry out other medical procedures at the target site, or another medical device besides the needle may be used for the procedure.

The needle 25 may include a proximal end that is configured to be deployed through the internal cavity of the tube 45; and a distal end that is configured to be connected to a vacuum for suctioning at the target site. The needle 25 may be configured to be steerable once inside the target site.

The needle 25 may be steered by at least one of the following means: a force from the distal end, such as, pushing the needle physically via , an inner wire inside an internal tube of the needle 25 that is configured to steer the needle 25 based on physician's operation or automatically using a computer guided process, a mechanical feature inside the needle 25 that receives and carries out an instruction(s) from a computer or a physician to change the needle 25's direction or angle, a hinge or any other mechanism inside the needle 25, or a combination thereof.

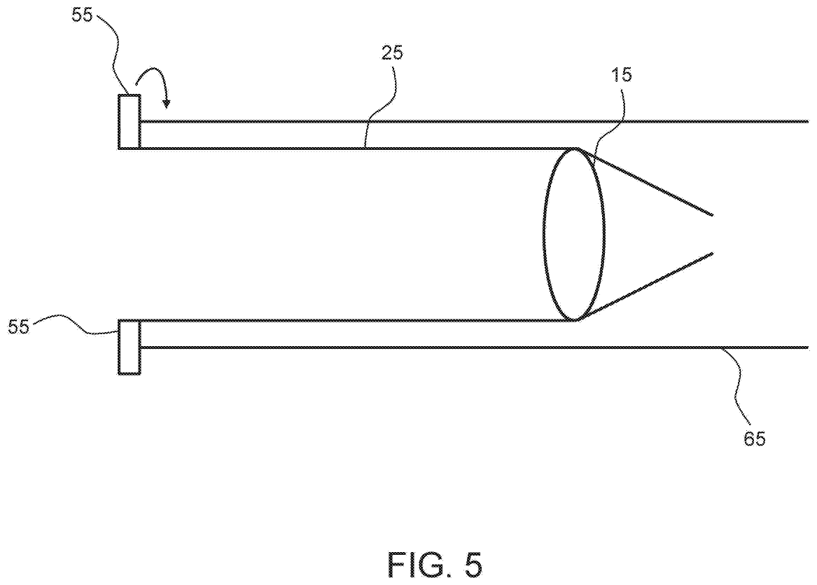

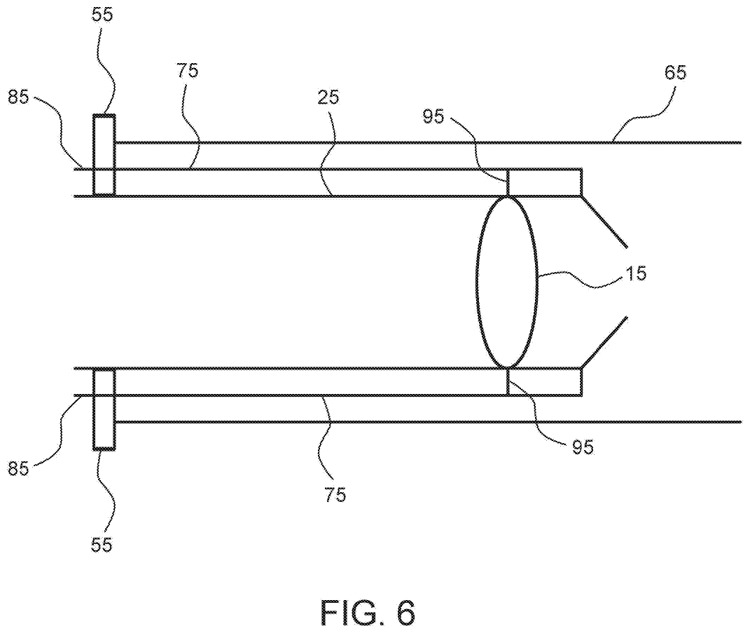

Referring to FIGS. 4 and 5 concurrently, the needle 25 may be covered by a protective sheath 65 so that the protective sheath 65 emerges from the internal channel/hole 47 and then the sheath 65 removed to reveal the needle 25. The needle 25 may also include a set of handle 55 that is connected to the elbow 15 as to move the tip of the needle 15 through movement of the handle 55. The handle 55 may be configured to be connected to, e.g., a machine, or may be controlled manually by a physician. The handle 55 may be rotated as to transmit the rotation to the needle tip. The handle 55 may also be used to change the direction of the needle tip. Alternatively, referring to FIGS. 4 and 6 concurrently, the endoscopic device may include a wire pulley 75 connected to the elbow 15 (and tip of the needle) by a pulley system 95, wherein the wire pulley 75 includes a tip 85 for controlling the pulley system 95. The tip 85 may be pulled/moved to change the direction and/or rotation of the needle 25's tip.

Referring to FIGS. 3-6 concurrently, the needle 25 may include a portion that is flexible at a mid-portion of the needle 25 and a portion that is not flexible at the proximal end so that the mid-portion may change its shape upon application of the above described means.

Alternatively, (or additionally), the proximal end of the needle 25 may be flexible as to allow the needle 25 to change its directions. The directional change may be less or more than 90 degrees. The handle 55 may be configured to be connected to a machine for operations or may be manually operated by a physician. The handle 55 may be configured to receive an instruction from a user (or a computer) and transmit said instruction to the needle and/or the guidewire such as, e.g., begin aspiration, deploy the guidewire/needle, carry out a medical procedure, move forward, backward, or sideways, and the like.

FIG. 4 illustrates another example of an endoscopic device for use inside a patient's body in accordance with the principles of the present disclosure. The endoscopic device includes a tube 45 with a proximal end and a distal end, the distal end coupled to a handle (or a suction tube) and the proximal end configured to enter into the patient's body; and an ultrasound probe (not shown) on the proximal end of the tube 45, positioned to emit sound waves to create a visual image of a target site inside the patient's body, wherein the tube 45 includes an internal cavity for a deployment of a needle 25. The needle 25 may include an internal cavity whereby a removable rod 35 may be fit inside. The removable rod 35 may include a plastic or metal and may be configured to provide a stabilizing force to the needle 25 so that the needle 25 maintains its shape until the needle 25 reaches a target site at which point the removable rod 35 is removed from the needle 25 so that the needle 25 can be steered or flexibly moved. The needle 25 may be deployed through the hole 47 on the tube (or a catheter) 45. The needle 25 may be stored inside the tube 45 until the tube 45 reaches a target site in vivo. Alternatively, the needle 25 may be inserted into the tube 45 through the tube 45's internal cavity after the tube 45 reaches a target site.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. An endoscopic device for use in a patient's body, comprising:
a tube having an internal cavity with a proximal end and a distal end, the distal end coupled to a handle and the proximal end configured to enter into the patient's body;
an ultrasound probe on the proximal end of the tube, positioned to emit sound waves to create a visual image of a target site inside the patient's body; and
a needle that is configured to be housed inside the internal cavity of the tube and configured to be deployed from the internal cavity, the needle defining a needle channel and including a distal section and a proximal section having a tip, and an elbow including a swivel ball joint configured to rotate up to 270 degrees or more in any direction such that the needle channel remains open.

2. The endoscopic device of claim 1, wherein the needle comprises at least one of: flexible metal, carbon fiber, plastic, or any other material that is non-toxic inside a human body.

3. The endoscopic device of claim 1, wherein the proximal section of the needle is configured to move freely along with a movement by the elbow.

4. The endoscopic device of claim 3, wherein the elbow is configured to be located at or near the proximal section of the needle.

5. The endoscopic device of claim 4, wherein the needle comprises multiple elbow joints with variable rotation angles and directions that are positioned proximal to the tip, and (c) a stiffening cannula configured to be placed inside the needle to avoid inadvertent rotation of the needle upon insertion into tissue, wherein depending on indication of needle insertion, the multiple elbow joints are placed more distal or proximal to the proximal end of the needle to have variable range of rotation.

6. The endoscopic device of claim 1, wherein the elbow comprises mechanistic features, such as, hinges, screws, fasteners, pulling mechanism, and other fastening mechanisms, that would allow the needle or a portion of the needle that protrudes from the elbow to change its angle and/or direction as needed or controlled by a physician or a system computer.

7. The endoscopic device of claim 1, wherein the elbow is further configured to be fixedly attached to the needle.

8. The endoscopic device of claim 1, wherein the ultrasound probe comprises an ultrasound transducer that emits and detects the sound waves to create the visual image of the target site inside the patient's body.

9. The endoscopic device of claim 8, wherein the needle, using the visual image for guidance, is directed to the target site.

10. The endoscopic device of claim 9, wherein the proximal section
is configured to be deployed through the internal cavity of the tube, and
the distal section is configured to be connected to a vacuum for applying suction at the target site through the proximal section of the needle.

11. The endoscopic device of claim 9, wherein the needle is configured to carry out aspiration at the target site.

12. The endoscopic device of claim 9, wherein the needle is steered by at least one of the following: (a) an inner wire inside an internal tube of the needle that is configured to steer the needle based on physician's operation or automatically using a computer guided process, (b) a mechanical feature inside the needle that receive an instruction from a computer or a physician to change the needle's direction or angle, (c) a hinge or any other mechanism inside the needle, (d) a pulley system that directs the needle's movement, or (e) a combination thereof.

13. The endoscopic device of claim 12, wherein the needle comprises a portion that is flexible at a mid-portion of the needle and a portion that is not flexible at the proximal section of the needle so that the mid-portion of the needle may change its shape upon steering the needle thereby changing the shape of the needle.

14. The endoscope device of claim 12, wherein the proximal section of the needle is configured to be flexible as to allow the needle to change its direction or angle.

15. The endoscopic device of claim 1, wherein the handle is configured to be connected to a machine for operations or manually operated by a physician.

16. The endoscopic device of claim 1, wherein the needle comprises an internal cavity that is configured to fit a removable rod inside the needle's internal cavity so that the needle maintains its shape.

17. The endoscopic device of claim 16, wherein the removable rod is further configured to provide a stabilizing force to the needle so that the needle maintains its shape until the needle reaches a target site inside the patient at which point the removable rod is removed from the needle so that the needle can be steered or flexibly moved.

18. A method for using an endoscopic device inside a patient's body comprising:
inserting the endoscopic device into a body cavity during an endoscopic surgical procedure, the endoscopic device including a needle with a distal section, a proximal section having a tip, and an elbow, the elbow including a fastener configured to fasten the distal section and the proximal section together;
steering the needle; and
carrying out an aspiration via the needle in order to collect a sample from the patient's body.

19. A system for carrying out an aspiration inside a patient's body comprising:
an endoscopic
device; a network;
a monitor computer;
a hosted server; and
a database,
wherein the network, the monitor computer, the hosted server, the database are coupled via communication links, and wherein the endoscopic device comprises (a) a tube with a proximal end and a distal end, the distal end coupled to a handle and the proximal end configured to enter into the patient's body, (b) an ultrasound probe on the proximal end of the tube, positioned to emit sound waves to create a visual image of a target site inside the patient's body, and (c) a needle that is configured to be housed inside the tube, wherein the tube includes an internal cavity for a deployment of the needle, the needle having an elbow including a ball joint configured to steer the needle.

20. The system of clam 19, wherein the endoscopic device, the monitor computer, the hosted server, and the database comprise a computer-readable medium tangibly embodying a computer readable program code having computer readable instructions which, when implemented, cause a computer to carry out the step of:
inserting the endoscopic device into a body cavity during an endoscopic surgical procedure; steering the needle; and carrying out an aspiration via the needle in order to collect a sample from the patient's body.
